(19) United States
(12) Patent Application Publication
(10) Pub. No.: US 2009/0005827 A1
(43) Pub. Date: Jan. 1, 2009

(54) Wearable defibrillator

(76) Inventors: David Weintraub, Yavne (IL); Yoram Eshel, Tel Aviv (IL)

Correspondence Address: EMPK & Shiloh, LLP;c/o Landon IP, Inc., 1700 Diagonal Road, Suite 450, Alexandria, VA, 22314, US

(21) Appl. No.: 11/819,197

(22) Filed: Jun. 26, 2007

(51) Int. Cl. A61N 1/39; A61H 31/00

(52) U.S. Cl. 607/6; 601/84

(57) ABSTRACT

There is provided a wearable defibrillator that includes a first sensor adapted to sense a cardiac related parameter, a second sensor adapted to sense breathing, a controller adapted to produce a signal upon determining a cardiac arrest and a defibrillating subunit adapted to provide a defibrillation energy upon receiving the signal from the controller. There is also provided a method of defibrillating that includes sensing a cardiac related parameter; sensing breathing; and triggering a defibrillation energy upon determining a cardiac arrest. There is further provided a wearable defibrillator that includes a first sensor adapted to sense a cardiac related parameter; a controller adapted to produce a signal upon determining a cardiac arrest; a transmitter adapted to send a signal indicative of a cardiac arrest to a remote location; and a defibrillation subunit adapted to trigger a defibrillation upon being activated from a remote location. In addition, there is provided a method of defibrillating that includes sensing a cardiac related condition; producing a signal upon determining a cardiac arrest; transmitting a signal indicative of a cardiac arrest to a remote location; and triggering a defibrillation upon being activated from the remote location.

### BACKGROUND

The vertebrate heart is an organ that pumps blood through the blood vessels and thus causes the blood to circulate throughout the body. Structurally, the heart, which is a muscular organ, is divided into four major compartments: right atrium, right ventricle, left atrium and left ventricle. Blood from the body (deoxygenated blood) enters the heart through the right atrium and moves to the right ventricle. From the right ventricle, the blood is pumped to the lungs. From the lungs, the blood (oxygenated blood) moves back to the left atrium of the heart. From the left atrium the blood transfer to the left ventricular, from which it is pumped to other parts of the body. The heart has an intrinsic ability to rhythmically contract and expend, a movement that creates the pumping capability of the heart. The contraction of the heart is a timely coordinated, rhythmic event. The contraction of the heart and flow of blood through the heart results in the production of a sound known as heart beat. The rhythmic contraction of the heart is caused by small electric currents that run through the heart in a cyclic manner. The small electric currents are initiated by intrinsic specialized heart cells and transferred along the heart walls. These specialized heart cells are localized in specific locations in the heart (such as the SA node) and are known as natural pace makers. These specialized cells produce electric currents that may directionally propagate throughout the heart and cause the synchronized rhythmic contraction of the heart. The electric activity of the heart may be detected by various methods, such as for example by a method known as electrocardiogram (ECG) that detects and records electrical activity of the heart.

Various abnormal heart conditions that may cause malfunctioning of the heart are well known. Most of these abnormal conditions may be life threatening. Some of the abnormal heart conditions may be attributed to faults in the electrical activity of the heart, which may lead to interference with the normal rhythmic contraction of the heart. Interferences of the normal rhythm of the heart, such as abnormal rhythm or abnormal heart rate are known as arrhythmia. The most common conditions of arrhythmias are: Tachycardia, Bradycardia and Fibrillation.

Tachycardia is a condition in which the heart beats at very high rates (more than 100 times per minute). Ventricular Tachycardia (VT) may result from interference of electrical currents generated in various locations of the heart. Bradycardia is a condition in which the heart beats at very slow rates (less than 60 beats per minute) and may result from a problem with the natural pacemaker or electrical conductance pathways of the heart. Fibrillation is a condition in which the heart fibrillates (twitches and quivers) in disorganized or desynchronized rhythms. Two kinds of Fibrillations are known: Atrial Fibrillation, in which the atrials beat at very high rates (300-600 beats per minute) and Ventricular Fibrillation (VF). Ventricular fibrillation is a condition in which the ventricles of the heart fibrillate (twitches and quivers) instead of contracting in a coordinated manner. As a result, blood pumping through the heart is disrupted. Ventricular fibrillation is a highly life threatening heart condition that may result is death within a very short period of time (in the order of 2-3 minutes), unless an external intervening is applied that would restore normal electrical activity to the heart.

Cardiac arrest is a most dangerous and lethal heart condition in which the heart does not pump blood and as a result blood circulation is disrupted. The main causes of cardiac arrest are ventricular fibrillation (VF), ventricular tachycardia (VT) and Pulmonary artery (aorta) blockage, with the arrhythmias conditions (VF and VT) being the most common causes of cardiac arrest. Clinically speaking, one can assume that a subject suffering from cardiac arrest suffers from VT/VF, even without the actual knowledge of it and start with defibrillation activity. In a case of real VT/VF the victim may be saved, while in cases other than that, a short defibrillation following by cardiac massage will not worsen the clinical situation.

Reinstating normal functioning of an arrhythmic heart may be performed for example by stimulating the heart, either by electrical means or mechanical means. Such stimulation may restore the heart its normal electrical activity and as a result, it's normal rhythm. Some of the methods known in the art that are routinely used in medical treatments include such methods as use of artificial pacemakers and defibrillation. Medical devices that are adapted to provide such treatments may apply these methods. These medical devices may be external devices or may be internal, implanted devices.

A defibrillator device is a device that is designed to restore a fibrillating heart its normal electrical activity, normal rhythm and hence it's normal activity and thus restore the patients life. Various kinds of defibrillator devices are known in the art both external defibrillators and internal defibrillators. External defibrillators are defibrillators that are placed externally on a patient in need and may be operated manually, for example by a trained medical stuff. Other external defibrillators may be operated automatically, for example by untrained bystanders. Some external defibrillators may also initiate the defibrillation process automatically. Such automatic external defibrillators may be further equipped or connected to a monitoring device (such as ECG) that may monitor the electrical activity of the heart and initiate a defibrillation process, when an abnormal electrical activity is detected. Some of the external defibrillators are designed to be mobile, for example to be carried in an ambulance, in hospitals or by individuals. Internal defibrillators are defibrillators that are implanted directly in the patient body. Such internal defibrillators are designed to actively monitor heart activity and are capable of immediately performing a defibrillation process upon detecting a condition of ventricular fibrillation, regardless of the patient's clinical condition (such as consciousness of the patient). Internal defibrillators are often in direct contact with the heart, both for monitoring purposes and for treatment purposes.

The defibrillation process is most often performed by electrical means, by which an electrical energy is delivered to the heart, either externally (such as for example by the use of electrodes or pads that are placed externally on the user body) or internally (such as for example by the use of electrodes that are in direct contact with the heart). The electrical energy applied to the heart may be monophasic (moving in one direction between the electrodes) or biphasic (moving in both directions between the electrodes). The electrical shock applied to the heart is measured in units of Joules and is designed to stop fibrillation of the heart, allowing for the restoration of normal rhythmic electrical activity of the heart. The defibrillation process may also be performed mechanically, for example by the use of a mechanical energy (chest thump) to the heart that may cause restoration of normal electrical activity of the heart. The mechanical energy may be applied for example by a direct-contact mechanical stimulus of the heart.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

An aspect of some embodiments relates to providing defibrillation.

According to an aspect of some embodiments, the defibrillation is performed upon sensing cardiac related parameters (such as pulse and/or electrical activity or the absence of it by mechanical or electrical means) and upon detecting, mechanically or electrically, a clinical sign that is directly or indirectly related to cardiac arrest, such as lack of breathing. The detection of a clinical sign that is directly or indirectly related to cardiac arrest (such as, for example lack of effective breathing and/or low/no blood pressure) in addition to sensing the cardiac related parameter (such as lack of pulse—no pulse is detected), may reduce or eliminate cases wherein defibrillation is performed based upon cases of VT/VF episode, which does not include a significant pulseless period (such as for example in a short run of ventricular tachycardia/ventricular fibrillation (VT/VF) episode, which does not include a significant pulseless period).

According to an aspect of some embodiments, the defibrillation is performed in a subject upon sensing a cardiac related parameter and upon receiving instruction to perform defibrillation from a remote location, for example by an expert such as a physician who approves that the subject is/was going through real and severe VT/VF episodes that otherwise deserve defibrillation and by remotely activating the system controller thus producing defibrillation. This may reduce or eliminate cases wherein defibrillation is performed too late based upon the time needed for the rescuers to arrive and perform defibrillation, as currently being the case.

There is provided, according to some embodiments, a wearable defibrillator that includes a first sensor adapted to sense a cardiac related parameter, such as electrical and mechanical cardiac pulse, a second sensor adapted to sense breathing, a controller adapted to produce a signal upon determining a cardiac arrest (the logical clinical situation that is determined by the controller, when there is no pulse and no breath detected) and a defibrillating subunit adapted to provide a defibrillation pulse upon receiving the signal from the controller.

There is further provided, according to some embodiments, a method of defibrillating that includes sensing a cardiac related parameter; sensing breathing; and triggering a defibrillation energy upon determining a cardiac arrest and upon determining lack of effective breathing.

There is further provided, according to some embodiments, a wearable defibrillator that includes a first sensor adapted to sense a cardiac related paremeter; a controller adapted to produce a signal upon determining a cardiac arrest; a transmitter adapted to send a signal indicative of a cardiac arrest to a remote location; and a defibrillation subunit adapted to trigger a defibrillation upon being activated from the remote location.

There is further provided, according to some embodiments, a method of defibrillating that includes sensing a cardiac related condition; producing a signal upon determining a cardiac arrest; transmitting a signal indicative of a cardiac arrest to a remote location; and triggering a defibrillation upon being activated from the remote location.

In addition to the exemplary aspects and embodiments described above, further aspects and embodiments will become apparent by reference to the figures and by study of the following detailed descriptions.

### BRIEF DESCRIPTION OF THE FIGURES

FIG. 1—Schematic block diagram of a wearable-defibrillator according to some embodiments;

FIG. 2—Schematic block diagram of carrying subunit according to some embodiments;

FIG. 3—Schematic block diagram of sensing subunit according to some embodiments;

FIG. 4—Schematic block diagram of defibrillation subunit according to some embodiments;

FIG. 5—Schematic block diagram of alarming subunit according to some embodiments;

FIG. 6—Schematic block diagram of controller subunit according to some embodiments; and

FIGS. 7 A,B,C—Schematic illustration of wearable-defibrillator according to some embodiments.

### DETAILED DESCRIPTION

In the following description, various aspects of the invention will be described. For the purpose of explanation, specific configurations and details are set forth in order to provide a thorough understanding of the invention. However, it will also be apparent to one skilled in the art that the invention may be practiced without specific details being presented herein. Furthermore, well-known features may be omitted or simplified in order not to obscure the invention.

According to some embodiments, there is provided a wearable defibrillating device. The wearable defibrillating device, referred herein as the “wearable-defibrillator” may include several subunits that may be functionally and/or physically interconnected. The subunits may include for example: a carrying subunit, a sensing subunit, a defibrillating subunit, a controller subunit, an alarming subunit, or any combination thereof. Illustrated in FIG. 1 a schematic block diagram of wearable-defibrillator (2) according to some exemplary embodiments. Further illustrated are various subunits of the wearable-defibrillator (2), such as: carrying subunit (100), sensing subunit (200), defibrillating subunit (300), alarming subunit (400) and controller subunit (500). The wearable defibrillator may be self activated or remotely activated.

### Carrying Subunit

The carrying subunit (such as carrying subunit 100 in FIG. 1) may include any carrying device that may be used to carry the wearable-defibrillator and hold it in a location chosen by the user. The carrying subunit may include any known carrying device or any combination of carrying devices that may be suited in size and function to carry the wearable-defibrillator. Some examples of carrying devices that may be used as carrying subunit are: pouch, garment, jacket, vest, pocket, sacks, compartments, belt or any combination thereof. The carrying subunit may be composed of various materials such as but not limited to: leather, plastic, fabric, stretchable fabric, elastic material, rubber or any combination thereof. The carrying subunit may be waterproof and may optionally be washable. The carrying subunit may be size adjustable to fit various body sizes of various users. The carrying subunit may be composed of a non-irritating material, such as hypoallergenic substance.

According to some embodiments, the carrying subunit may include a vest. The vest may be fitted to carry the wearable-defibrillator various subunits. The vest may be fitted, for example, with pockets, pouches, compartments, and any combination thereof. The vest may be adjustable in size to fit various users body sizes. The vest may optionally be washable.

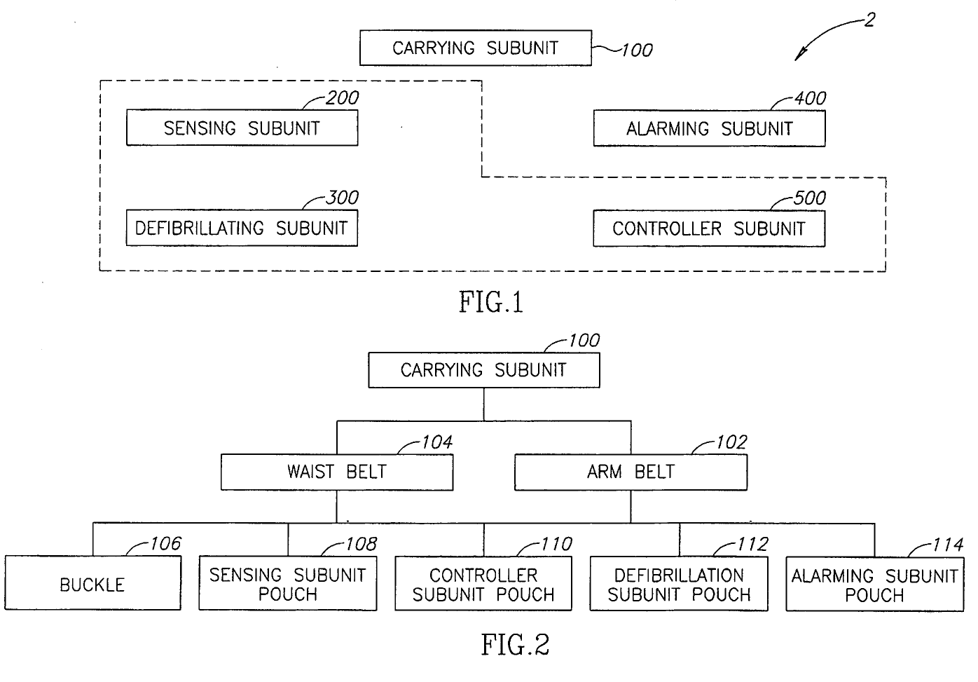

According to some preferred embodiments, the carrying subunit may include a belt, termed herein “carrying belt” that may be used to carry and hold the wearable-defibrillator and its various subunits in place. Reference is now made to FIG. 2, which illustrates a block diagram of a carrying subunit according to some preferred embodiments. The carrying subunit (such as carrying subunit 101 in FIG. 2), may be composed of various materials such as leather, plastic, fabric, stretchable fabric, elastic material, rubber or any combination thereof and may further include buckles of various sorts, zippers and the like, used to lock the subunit in place. The carrying subunit may be waterproof and may optionally be washable. The carrying subunit may be size adjustable. The carrying subunit may be composed of two belts: the “arm belt” (102) and the “waist belt” (104) that may be in direct contact or separated. The arm belt (102) may be worn, for example, around the user left shoulder. The waist belt (104) may be worn, for example, around the user's waist. The carrying subunit may contain several subassemblies that are used to carry and position the various subunits of the wearable-defibrillator. The carrying subunit may include subassemblies such as pockets, pouches, slots, buckles, zippers or any combination thereof. The subassemblies may be an integral part of the carrying subunit; for example, the subassemblies may be directly sawn to the carrying subunit. Alternatively, the subassemblies may be reversely connected to the carrying subunit by various means, such as buttons, zipper, adhesive stripes, Velcro and the like, or any combination thereof. The subassemblies may be composed of various materials such as leather, plastic, fabric, stretchable fabric, elastic material, rubber or any combination thereof and may optionally be waterproof. For example, the carrying subunit may integrally include pouches for the various other subunits of the wearable-defibrillator, such as pouch for a sensing subunit (108), pouch for an alarming subunit (114), pouch for a defibrillation subunit (112), and pouch for a controller subunit (110). The waist belt may further include buckle(s) (such as buckle 106) that are used to close and lock the belt in place. The belt buckle may further include a sensing element that may indicate that the buckle is closed; hence, the belt is closed and secured to the user's body.

### Sensing Subunit

The sensing subunit of the wearable-defibrillator is a subunit that contains the sensing elements that are referred to herein as “sensors”. According to some embodiments, the sensors may be used to sense/measure physiologically related parameters. The sensors may further include non-physiologically related sensors that are used to sense/measure mechanical parameters that are related to the operation of the wearable-defibrillator device. The data sensed by the sensors is collected and transferred by the sensing subunit to the controller subunit. In addition, the sensing subunit may include a power source used for activating and operating the sensing subunit. The sensing subunit may further include an indicator element that may provide indication related to various parameters measured by the sensors of the sensing subunit.

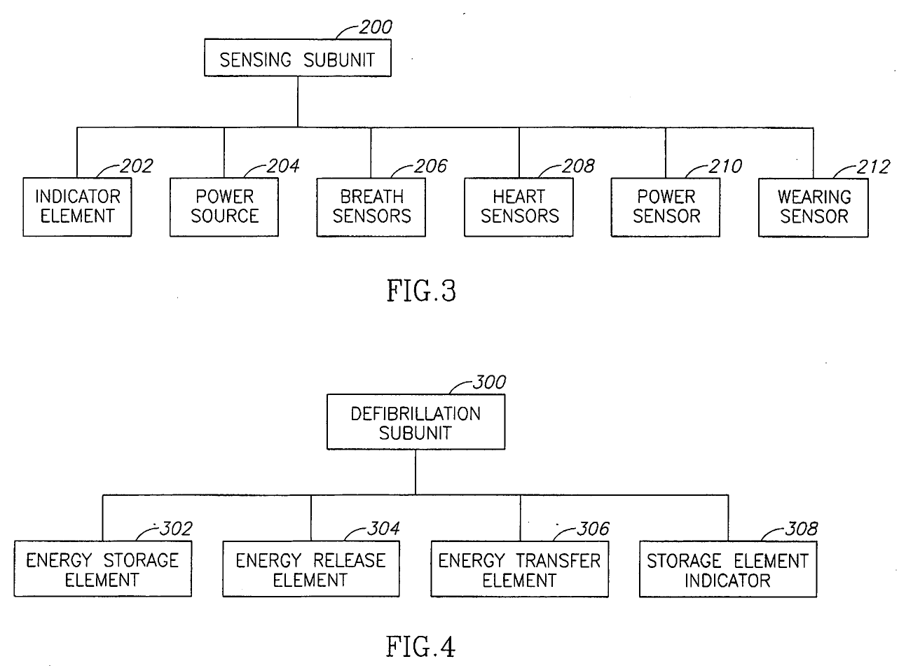

Reference is now made to FIG. 3, which illustrates a sensing subunit according to some embodiments. The sensing subunit (such as sensing subunit 200 in FIG. 3), may be a closed, waterproof subunit. The sensing subunit (200) may operate on power obtained from an internal power source (such as power source 204) that is located within the sensing subunit. The internal power source may include, for example one or more rechargeable batteries that may be located within a battery compartment situated within the sensing subunit. The rechargeable batteries may include any kind of rechargeable batteries that are known in the art, such as, but not limited to Li-Ion, Ni—Me, Ni—Cd. The rechargeable batteries may have a battery life span of at least 5 years and the batteries may be recharged by connecting to an external power source, such as an electric power network. The rechargeable batteries may be user replaceable. Alternatively, the rechargeable batteries may not be accessible for replacement by a user, as not to impair the waterproofing of the closed sensing subunit (200).

According to some embodiments, the sensor subunit (200) may include sensors used to sense and/or measure various parameters that are directly related to the operation of the wearable-defibrillator (such as wearable-defibrillator 2 in FIG. 1). In addition, the sensor subunit may include sensors that may sense/measure physiologically related parameters. The physiologically related parameters may include, for example, breath and breath related parameters (measured by “breath sensors”, such as breath sensors 206 in FIG. 3), blood related parameters (measured by “blood sensors”), heart functioning related parameters (measured by “heart sensors”, such as heart sensors 208 in FIG. 3), movement sensors, or any combination thereof. The sensors may include electromechanical sensors, electrical sensors, mechanical sensors or any combination thereof that may be adapted to sense the physiologically related parameters to be measured. For example, measurement of breath and breath related parameters may be performed for example by pressure sensor. The pressure sensor may sense pressure variations of the thorax, which are indicative of breathing and an effective breathing. The pressure sensor may include, for example, one or more piezo electrodes that may be placed on the user thorax and able to sense variation in pressure by the thorax, caused by the movement of the thorax during breathing cycles of inhalation and expiration. The breathing sensors may include, for example, strain gage sensors. The breath related parameters may also be measured using a sonic sensor. The sonic sensor may sense sound caused by air movement during the inhalation and exhalation stages of the breath cycle. Blood related parameters may include for example, blood pressure, blood oxygenation, or any other blood related parameter that may be used as an indicative of the activity of heart. Blood related parameters may be measured by various sensors and subassemblies that may be fitted to the sensing subunit of the wearable-defibrillator device. Sensors and subassemblies used to measure blood related parameters may include any known blood monitoring device such as for example non-invasive blood pressure monitoring device, non-invasive blood oxygenation monitoring device or any combination thereof. Heart functioning related parameters may include for example heart beat (heart pulse) and beat to beat variation. Measurement of heart pulse may be performed by various sensors. For example, a sonic sensor placed on the user thorax in proximity to the location of the heart may sense the sonic sound of the heart beat and thus give an indication as to the functioning of the heart. A pressure sensor placed externally on the user's thorax in proximity to the user's heart may be used to mechanically sense the pulse of the heart. An example of a pressure sensor may be a piezo electric sensor. One or more piezo electrodes may be placed externally on the user's thorax in close proximity to the heart. The piezo electrode may then sense the localized pressure changes caused by the pulsing of the beating heart. Measurement of heart pulse/heart activity may be performed, for example, by measuring electrical activity of the heart. Measuring electrical activity of the heart may be performed, for example by the use of electrocardiograph. The electrocardiograph may record the electrical activity of the heart and produce an electrocardiogram (ECG), which presents a graphical display of the heart electrical activity and provides an indication for the heart overall activity and heart pulse. The electrocardiograph sensors may include leads that are connected to the user body at specific locations and are used to sense electrical activity of the heart. The electrical heart sensors may be used for example, to detect ventricular tachycardia/ventricular fibrillation (VT/VF) episodes. In addition to measuring individual heartbeats by any method described aboveherein, the heart sensors may be further adapted to measure beat to beat variation. Beat to beat variations are naturally occurring beat-to-beat changes in heart rate. Under normal physiological conditions, beat-to-beat variation may be detected. However, lack or decline of beat to beat variation, may be indicative of an abnormal pathological physiological condition. For example, beat-to-beat variation may be influenced by breathing. When breathing is impaired, beat-to-beat variation decline and may not be detected. In order to measure beat to beat variations, the heart sensors may be further equipped with a timing device and a processor that may be adapted to track changes in heart rate, by timing the heart beats over a period of predetermined time, such as, for example, 15-120 seconds.

According to some embodiments, the sensing subunit may further include additional sensors that are adapted to measure parameters that are related to the operation of the wearable-defibrillator. The sensors may include electrical sensors, mechanical sensors, electromechanical sensors, or any combination thereof. The parameters that are related to the operation of the wearable-defibrillator may include, for example, power source charging level (“power sensor”, such as power sensor 210 in FIG. 3), sensing correct wearing of the device by the user (“wearing sensor”, such as wearing sensor 212 in FIG. 3). For example, the power sensor may be used to measure the level of charging of the sensing subunit power source.

According to some embodiments, the sensing subunit may further include an indicating element (such as indicating element 202 in FIG. 3) that may present indications regarding proper operation of various sensors. For example, the indicating element may present indications that the breathing (and/or blood) sensors and pulse sensor are operative and that breathing and pulsing is being detected by the sensors. The indicating element may further provide indications regarding the power source status (such as for example, level of charging). The indicating element may further provide indication that the wearable-defibrillator is properly placed on the user body (as indicated, for example, by the “wearing sensor”). The indications provided by the indicating element may include for example, visual indications, sonic indications or any combination thereof.

According to some preferred embodiments, the indications provided by the indicating element (214, FIG. 3) of the sensing subunit of the wearable-defibrillator may include lights of various colors. For example, a set of green and red lights are provided for breathing detection wherein green light turned on indicates that breathing is being detected by the sensors while red light turned on indicates that no breathing is being detected. Likewise, a set of green and red lights are provided for pulse detection wherein green light turned on indicates that heart beat is being detected by the sensors while red light turned on indicates that no heart beats are being detected. In addition, a set of green and red lights are provided for indicating the status of the power source, wherein green light turned on indicates power source is charged to full capacity and red light turned on indicates power source levels are low, such as for example at only about 15% of maximal capacity. An additional indicating green light may be directed to indicate that the wearable-defibrillator is properly placed on the user body.

According to some embodiments, the information measured/sensed by the various sensors of the sensing subunit may be transferred by various means to the controller subunit of the wearable-defibrillator. The information measured/sensed by the sensors may be transferred to the controller subunit by any known communication route, either by direct contact, such as for example via wires, or by indirect contact, such as for example by wireless communication. Each sensor may transfer the information to a corresponding element within the sensing input module located within the controller subunit. The information from the sensing subunit may be sent continuously and instantly.

### Defibrillation Subunit

The defibrillation subunit is a subunit that may activate the defibrillation process upon a given cue. The cue may be issued internally by the defibrillation subunit, or may optionally be issued externally from a remote location. According to some embodiments, and as illustrated in FIG. 4, the defibrillation subunit (such as defibrillation subunit 300) may include various elements that may be used to initiate and perform the defibrillation process. The defibrillation subunit may include such elements as, but not limited to energy storage element (302), energy transfer element (306), energy release element (304), indicator element (308), or any combination thereof. The various elements of the defibrillation subunit may be physically and/or functionally interconnected, either by direct contact (for example via wires), or not in direct (for example, via wireless connection).

According to some embodiments, the defibrillation subunit may include an energy storage element (such as energy storage element 302 in FIG. 4). The energy storage element is an element that may have the capacity to store amount of energy, such as for example electrical energy, mechanical energy or any other applicable form of energy that may be stored. The energy storage element may be charged with energy and store the energy within, until an activating signal causes discharging of the stored energy. The charging of the energy storage element may be performed by external intervening, for example by the user, upon placing of the wearable-defibrillator device, or may be performed with no external intervening, upon a given cue. According to some exemplary embodiments, the energy stored within the energy storage element may be for example electrical energy, and the storage element may include, for example an internal network of capacitors that may be charged by a power source and store the energy within. Charging the energy storage element with electrical energy may be performed by external intervening, for example by a user that may connect the energy storage element to an external power source. Optionally, the charging of the energy storage element with electrical energy may be performed without external intervening, for example it may be performed by the wearable-defibrillator device, either at a temporal stage wherein the wearable-defibrillator is being placed on the user body, or in close temporal proximity to the time the energy stored in the energy storage element is to be discharged. The energy stored in the energy storage element may include, for example mechanical energy. The mechanical energy may be stored, for example in a spring that may be charged by being wired. Wiring of the spring may be performed externally, for example, by the user that may wire the spring to its coiled position.

The defibrillation subunit may further include a storage element indicator (such as storage element indicator 308 in FIG. 4). The storage element indicator may indicate the level of charging of the energy storage element. The storage element indicator may include visual indications, sonic indications, or any combination thereof. The storage element indicator may indicate percentage of charged storage capacity (such as between 0-100% of charged storage capacity). The storage element indicator may indicate only conditions of empty and/or full storage capacity charge.

The defibrillation subunit may further include an energy release element (such as energy release element 304 in FIG. 4). The energy release element may receive an activating signal from the controller subunit of the wearable-defibrillator. Once activated, the energy release element may communicate with the energy storage element (302) and cause release of energy stored within the energy storage element (302). The defibrillation subunit may further include energy transfer element (306). The energy transfer element may include for example, at least two electrodes, pads or any other means of energy delivery. In case of transfer of electrical energy, electrodes may be used and may include, for example, high voltage electrodes. In case of transfer of mechanical energy, an impact pad may be used to deliver an external physical impact to the heart. The energy transfer element may be externally placed on the user body (such as for example on the user's skin). The energy transfer element may be placed at several positions. For example, the energy transfer element (306) may be positioned at anterior position and/or posterior position that are correlated with the location of the heart. The energy transfer element (306) may be directly connected to the energy release element (304). The energy release element (304) may transfer the energy stored in the energy storage element (302) to the energy transfer element (306). The energy release element may transfer any amount of energy to the energy transfer element, either amount that corresponds to the full capacity stored within the energy storage element, or partial amount of that energy. The amount of energy transferred to the energy transfer element may be predetermined or instantly determined, and may be constant or variable. The energy transferred to the energy transfer element may be for example electrical energy. The electrical energy transferred to the energy transfer element may be expressed in joules. For example, the electrical energy transferred may be for example in the range of 50 to 400 Joules. The electrical energy transferred may be transferred as a monophasic current, wherein the electrical energy current delivered is monodirectional, meaning it may move in one direction (for example, in a direction from the positive towards the negative ends of the energy transfer element). The electrical energy transferred may be transferred as a biphasic current, wherein the electrical current delivered may move in two directions (for example, moving from the positive towards the negative and from the negative towards the positive ends). Upon energy transfer from the energy release element to the energy transfer element, energy may be transferred to the users body. The energy transferred through the energy transfer element to the user's body may be localized and may occur at the contact regions between the energy transfer element and the user's body. The energy transfer from the energy transfer element to the user's body may occur instantly after energy is transferred from the energy release element to the energy transfer element. The transfer of energy to the energy transfer element may occur any number of times that may be predetermined or determined according to information processed by the controller subunit. The energy transfer may be performed autonomously by the wearable-defibrillator device, or may be performed by external activation of the wearable-defibrillator. External activation of the wearable-defibrillator may be performed for example from a remote location. The remote location may be, for example, a health care clinic that may receive information from the wearable-defibrillator (for example, by cellular communication route). Activation of the wearable-defibrillator may be initiated, remotely (for example, by cellular communication means) by a health care provider, situated within the remote location.

### Alarming Subunit

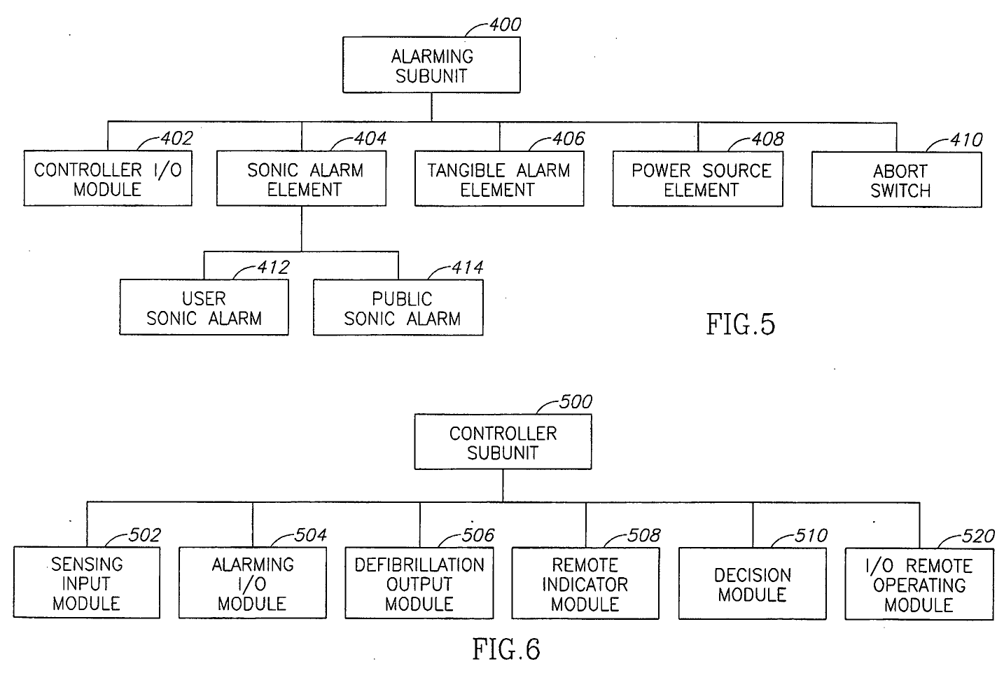

The alarming subunit is a subunit that may issue an alarm upon a given cue. For example, the alarming subunit may issue an alarm prior to activation of the defibrillation subunit. The alarming subunit may receive information from the controller subunit, issue an external alarm and within a period of time sends a response to the controller subunit. According to some embodiments, as illustrated in FIG. 5, the alarming subunit (400) may include several modules and elements, that may be functionally and/or physically interconnected, such as, but not limited to: controller I/O module (402), Sonic alarm element (404), tangible alarm element (406), power source element (408), On/Off element (410) or any combination thereof.

According to some embodiments, the alarming subunit (400) may include an input/output module that is termed herein “controller I/O module” (402). The controller I/O module (402) may receive and send information from and to the controller subunit (such as controller subunit 500 in FIG. 1). In addition, the controller subunit may send information to other modules and elements of the alarming subunit. The controller I/O module (402) may receive an activating signal from the controller subunit (500, FIG. 1). When an activating signal sent from the controller subunit is received by the controller I/O module, the controller I/O module may then send an activating signal to the sonic alarm element (404) and the tangible alarm element (406). The sonic alarm (404) may consist of two separate sonic alarms, the “user sonic alarm” (412) and the “public sonic alarm” (414), that may each be directed to alert different audience. The sonic alarm (404) may include any sound, noise, recorded voice massage or any combination thereof. The user sonic alarm (414) may be directed to alert the user of the wearable-defibrillator. Alerting the wearable-defibrillator user may be performed, for example, by noise, sound, such as high pitch alarming beeping sound, prerecorded alarm massage, or any combination thereof, and its aim is to alert to user that a defibrillation is about to be initiated. The user sonic alarm may be used to prevent defibrillation to a user who is not is need for such a process, as unnecessary defibrillation to a user may cause irreversible damage to the user. The public sonic alarm (414) may be preformed by prerecorded warning massage, noise, sound or any combination thereof and its aim is to alert potential audience, such as bystanders to move from the user since defibrillation is about to be initiated. The need for the public sonic alarm (414) arises because the defibrillation process may affect bystanders that are in direct contact with the user that is being defibrillated. According to some embodiments, the tangible alarm element (406) may be used to produce an alarm that would palpate the user, for example by stimulating the user skin. The tangible alarm (406) may include for example small electrical currents that may be produced by the tangible alarm element. The small electrical currents may be used, for example, to stimulate the user skin. The aim of the tangible alarm is to attract the user attention and notify that a defibrillation process is about to be initiated. Alerting a user that defibrillation is about to occur is designed to prevent defibrillation of a user who is not is need of such defibrillation.

According to some embodiments, the alarming subunit (400) may further include an On/Off element, termed herein “Abort switch” (410). The abort switch (410) may include any switch that may be operated by the user, and is designed to allow the user to turn off the alarm. Once operated by the user, the abort switch (41) turns off the sonic alarm (404) and the tangible alarm (406). In addition, the abort switch (410) may send a signal (“Abort signal”) to the controller I/O module (402). The controller I/O module may then output the abort signal to the alarming I/O module of the controller subunit, which would then stop the upcoming defibrillation process.

### Controller Subunit

The controller subunit is a subunit that may receive input from other subunits of the wearable-defibrillator, process the information received and outputs orders to various other subunits of the wearable-defibrillator. According to some embodiments and as illustrated in FIG. 6, the controller subunit (500) may include several modules that may be functionally and/or physically interconnected, either by direct contact, (for example, by wires), wireless or any combination thereof. The controller subunit (500) may include for example such modules as: input module (502) that receives information from the sensing subunit, input/output module (504) that communicates with the alarm subunit, output module (506) that communicates with the defibrillation subunit, decision module (510) that may process the information received from the various subunits. The controller subunit (500) may further include a remote indicator module (508) that may be used to send an emergency call to a predetermined number. In addition, the controller subunit may further include an I/O remote operating module (520) that may send information to a remote location and receive an input signaling from the remote location.

According to some embodiments, the controller subunit (500) may include an input module that may receive information from the sensing subunit and is termed herein “sensing input module” (502). The sensing input module (502) may receive information that includes data and measurements that are being sent from the sensing subunit (200, FIG. 3). The information sent from the sensing subunit may be sent continuously and instantly. The information being sent may include, for example, data/measurements sent from the breath sensors (206, FIG. 3), blood sensors, heart pulse sensors (208, FIG. 3), wearing sensors (212, FIG. 3), power sensors (210, FIG. 3) or any other sensor operative in the sensing subunit (200, FIG. 3). The sensing input module (502) may include a receiving element that may receive information from all sensors. The sensing input module may include separate receiving elements that may each be adapted to receive information from a corresponding sensor. The information received from the breath sensors (206, FIG. 3) may include for example indication that breathing is detected by the breath sensor, number of breaths per minute that is being measured by the breath sensor or any other breath related information that is being sensed/measured by the breath sensors. The information received from the blood sensors may include for example indication that blood parameters, such as blood pressure and blood oxygenation that are being detected by the blood sensors are within a normal predetermined range. Likewise, the information received from the heart sensors (208, FIG. 3) may include, for example indication the heart pulse is being detected, number of heart beat per minute, electrical activity of the heart, rhythmic contraction of the heart, fibrillation of the heart, beat to beat variation, or any other heart related information that is being sensed/measured by the heart sensors. Information received from the wearing sensor (212, FIG. 3) may include, for example, indication that the wearable-defibrillator is properly placed and secured to the user's body. Alternatively, the wearing sensor may send indication that the wearable-defibrillator is not “in-use”, meaning it is not placed on the user's body. Information received from the power sensors (210, FIG. 3) may include, for example, the recharging status/level of the power source located in the sensing subunit, as sensed by the power sensor. The transfer of information from sensing subunit and the controller sensing input module may be performed via any known communication route, such as wired or wireless communication.

According to some embodiments, the controller subunit (500) may further include an input/output module to the alarming subunit, termed herein “alarming I/O module” (504). The alarming I/O module (504) may send and receive information from the alarming subunit (such as alarming subunit 400 in FIG. 4) of the wearable-defibrillator. For example, the alarming I/O module (504) may send information to the alarming subunit. The alarming I/O module (504) may further receive information sent from the alarming subunit (400, FIG. 4). The transfer of information from and to the alarming I/O module may be performed via any known communication route, such as wired or wireless communication.

According to some embodiments, the controller subunit (500) may further include an output module to the defibrillation subunit, termed herein “defibrillation output module” (506). The defibrillation output module sends information to the defibrillation subunit (300, FIG. 4) of the wearable-defibrillator. The information sent by the defibrillation output module (506) may include, for example, instructions to activate the defibrillation subunit (300, FIG. 4). The transfer of information to the defibrillation output module may be performed by sending information via any known communication route, such as wired or wireless.

According to some embodiments, the controller subunit (500) may further include a remote indicator module (508). The remote indicator module (508) may be used to send an emergency call to a predetermined number. The emergency call may be performed by cellular means, or any other applicable way of communication. The remote indicator module (508) may be activated to send an emergency call only in conditions wherein the defibrillator subunit was activated by instruction sent from the defibrillation output module (506). The transfer of information to the remote indicator module may be performed by sending information via any known communication route, such as wired or wireless.

According to some embodiments, the controller subunit (500) may further include a remote I/O operating module (520). The remote I/O operating module (520) may be used to initiate communication with a remote location and send and receive information from the remote location. The communication between the remote I/O operating module and the remote location may be performed by any known communication route, such as wired or wireless. For example, communication may be performed by cellular means, by initiating a call to the remote location. The remote location may predetermined and may include for example, a health care provider clinic, a hospital, a health care center. The information sent to the remote location may include information that is processed by the decision module (510), as detailed herein below. A health care provider (such as a medical doctor, intern, nurse and the like) that is situated at the remote location may review the information received from the remote I/O operating module in real time and upon reviewing the information, may send information to the remote I/O operating module. For example, information sent by the health care provider may include instructions to initiate a defibrillation process.

According to some embodiments, the controller subunit may further include a decision module (510). The decision module (510) is a module that may receive information from various other modules of the controller subunit (500), process the information and issue instructions to various other modules of the controller subunit and thus, indirectly issue instructions to the various other subunits of the wearable-defibrillator. The decision module (510) may include a microprocessor that may receive information from various controller modules, process the information and issue instructions to various controller modules and subunits of the wearable-defibrillator. The decision module (510) may receive information from various controller modules simultaneously or non-simultaneously, instantly or in predetermined time intervals, such as every 15-30 seconds. For example, the decision module (510) may receive information from the sensing input module and upon processing the information makes a decision whether instruction are to be output to other modules. The decision module may further receive information from the alarming subunit (400, FIG. 5) and upon processing the information makes a decision whether instructions are to be output to other modules. The transfer of information to and from the decision module may be performed by any known communication route, such as wired or wireless.

According to some exemplary embodiments, the decision module (510) may receive information from the sensing input module (502). If the information sent from the sensing input module (502) to the decision module (51) indicates that, with in a predetermined period of time, such as 15-60 seconds, no breathing is detected, no heart pulsing (cardiac arrest) is detected and the wearing sensor indicates that the wearable-defibrillator is in use; an activation signal is issued to the alarming I/O module (504). The alarming I/O module would then output a signal to the alarming subunit. If no information is being sent back from the alarming subunit (such as for example, “abort signal”) to the alarming I/O module (504) of the controller subunit, within a predetermined period of time (such as between 15-45 seconds), an activating signal may be sent from the decision module (510) to the defibrillation output module (506). The defibrillation output module (506) may then issue an output signal to the defibrillation subunit (300, FIG. 4), whereby the defibrillation subunit would be activated. Activation of the defibrillation process may be performed several times, as needed until normal heart activity is restored. In addition, the decision module (510) may activate the remote indicator module (508) to initiate an emergency call. All information transfer between the various modules may be performed via any communication route, such as wireless communication, wired communication, for example by the use of wires that interconnect the various modules and subunits, or any combination thereof.

According to some exemplary embodiments, the decision module (510) may receive information from the sensing input module (502). If the information sent from the sensing input module (502) to the decision module (51) indicates that, with in a predetermined period of time, such as 15-60 seconds, no breathing is detected, non rhythmic contraction of the heart (heart fibrillation) is detected and the wearing sensor indicates that the wearable-defibrillator is in use; an activation signal is issued to the alarming I/O module (504). The alarming I/O module would then output a signal to the alarming subunit. If no information is being sent back from the alarming subunit (such as for example, “abort signal”) to the alarming I/O module (504) of the controller subunit, within a predetermined period of time (such as between 15-45 seconds), an activating signal may be sent from the decision module (510) to the defibrillation output module (506). The defibrillation output module (506) may then issue an output signal to the defibrillation subunit (300, FIG. 4), whereby the defibrillation subunit would be activated. Activation of the defibrillation process may be performed several times, as needed until normal heart activity is restored. In addition, the decision module (510) may activate the remote indicator module (508) to initiate an emergency call. All information transfer between the various modules may be performed via any communication route, such as wireless communication, wired communication, for example by the use of wires that interconnect the various modules and subunits, or any combination thereof.

According to some exemplary embodiments, the decision module (510) may receive information from the sensing input module (502). If the information sent from the sensing input module (502) to the decision module (51) indicates that, with in a predetermined period of time, such as 15-60 seconds, no breathing is detected, no heart pulsing (cardiac arrest) is detected and the wearing sensor indicates that the wearable-defibrillator is in use; an activation signal is issued to the alarming I/O module (504). The alarming I/O module would then output a signal to the alarming subunit. If no information is being sent back from the alarming subunit (such as for example, “abort signal”) to the alarming I/O module (504) of the controller subunit, within a predetermined period of time (such as between 15-45 seconds), an activating signal may be sent from the decision module (510) to the remote I/O operating module (520). The remote I/O operating module (520) may then initiate a connection with a designated remote location and send the information from the decision module to the remote location. The remote location may include, for example, a health care clinic. A health care provider, situated in the remote location may then urgently review in real time the information sent by the remote I/O operating module (520). If the health care provider decides a defibrillation is in need, he may send an activating signal to the remote I/O operating module (520). The remote I/O operating module (520) may then activate the defibrillation output module (506). The defibrillation output module (506) may then issue an output signal to the defibrillation subunit (300, FIG. 4), whereby the defibrillation subunit would be activated. Activation of the defibrillation process may be performed several times, as needed until normal heart activity is restored. All information transfer between the various modules may be performed via any communication route, such as wireless communication, wired communication, for example by the use of wires that interconnect the various modules and subunits, or any combination thereof.

According to some exemplary embodiments, the decision module (510) may receive information from the sensing input module (502). If the information sent from the sensing input module (502) to the decision module (51) indicates that, with in a predetermined period of time, such as 15-60 seconds, no breathing is detected, non rhythmic contraction of the heart (heart fibrillation) is detected and the wearing sensor indicates that the wearable-defibrillator is in use; an activation signal is issued to the alarming I/O module (504). The alarming I/O module would then output a signal to the alarming subunit. If no information is being sent back from the alarming subunit (such as for example, “abort signal”) to the alarming I/O module (504) of the controller subunit, within a predetermined period of time (such as between 15-45 seconds), an activating signal may be sent from the decision module (510) to the remote I/O operating module (520). The remote I/O operating module (520) may then initiate a connection with a designated remote location and send the information from the decision module to the remote location. The remote location may include, for example, a health care clinic. A health care provider, situated in the remote location may then urgently review in real time the information sent by the remote I/O operating module (520). If the health care provider decides a defibrillation is in need, he may send an activating signal to the remote I/O operating module (520). The remote I/O operating module (520) may then activate the defibrillation output module (506). The defibrillation output module (506) may then issue an output signal to the defibrillation subunit (300, FIG. 4), whereby the defibrillation subunit would be activated. Activation of the defibrillation process may be performed several times, as needed until normal heart activity is restored. All information transfer between the various modules may be performed via any communication route, such as wireless communication, wired communication, for example by the use of wires that interconnect the various modules and subunits, or any combination thereof.

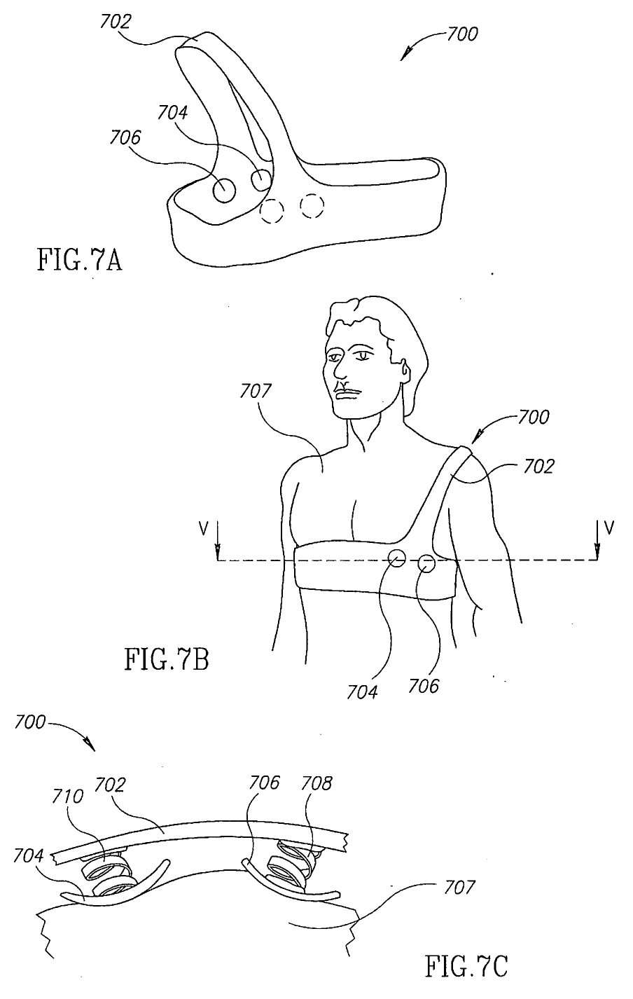

Reference is now made to FIG. 7, which illustrates a wearable defibrillator according to some embodiments. FIG. 7A illustrates a back view of a wearable defibrillator 700. The wearable defibrillator 700 illustrated in FIG. 7A may include, among others, a carrying subunit, such as carrying subunit 702 and a defibrillation subunit that may include, among others, energy transfer elements, such as energy transfer element 704 and energy transfer element 706. FIG. 7B illustrates a front view of a wearable defibrillator, according to some embodiments. The wearable defibrillator, 700, may include, among others, a carrying subunit, 702, that may be used to place the wearable defibrillator to a user, such as user 707, illustrated in FIG. 7B. The wearable defibrillator may further include a defibrillation subunit that may include, among others, energy transfer elements that are used to transfer defibrillation energy to the user, such as energy transfer element 704 and energy transfer element 706, illustrated in FIG. 7B. FIG. 7C illustrates a top view cross section of a wearable defibrillator according to some embodiments. The top view cross section illustrated in FIG. 7C is taken along line v-v of FIG. 7B. As Shown in FIG. 7C, the wearable defibrillator, 700, may include a carrying subunit (702) used to place the wearable defibrillator to the user (707). The wearable defibrillator may further include, among others, a defibrillating subunit that may include, energy transfer elements, such as energy transfer elements 704 and 706. The energy transfer elements, such as energy transfer elements 704 and 706 may be located in close proximity, and more preferably, in direct contact with the user body (707). The defibrillation subunit of the wearable defibrillator (700) may further include, among others, energy storage elements, such as energy storage elements 708 and 710 shown in FIG. 7C. Energy storage elements, 708 and 710, illustrated in FIG. 7C, demonstrate an exemplary mechanical energy-storage elements, wherein the mechanical energy may be stored, for example, in a spring that may be charged by being wired. The energy storage elements, 708 and 710 may be directly connected to the energy transfer elements 706 and 704.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

What we claim is:

1. A wearable defibrillator comprising:
a first sensor adapted to sense a cardiac related parameter;
a second sensor adapted to sense breathing;
a controller adapted to produce a signal upon determining a cardiac arrest upon sensing lack of effective breathing with no pulse; and
a defibrillating subunit adapted to provide a defibrillation energy upon receiving the signal from said controller.

2. The wearable defibrillator of claim 1, wherein said first sensor is adapted to sense mechanical or electrical pulse, obtain a electrocardiogram (ECG), or a combination thereof.

3. The wearable defibrillator of claim 1, wherein controller is further adapted to determine lack of breathing base on a cardiac related signal, obtained from said sensor.

4. The wearable defibrillator of claim 1, wherein said first sensor comprises an electric sensor, an electromechanical sensor or both.

5. The wearable defibrillator of claim 4, wherein said electromechanical sensor comprises a piezo electric sensor.

6. The wearable defibrillator of claim 4, wherein said electromechanical sensor comprises strain gage sensor.

7. The wearable defibrillator of claim 4, wherein said electromechanical sensor comprises a pressure sensor.

8. The wearable defibrillator of claim 7, wherein said defibrillating energy is an electrical energy.

9. The wearable defibrillator of claim 1, wherein said defibrillating energy is a mechanical energy.

10. The wearable defibrillator of claim 1, further comprising an alarming subunit adapted to trigger an alarm upon receiving an alarm signal from a controller, wherein the controller is adapted to produce an alarm signal upon receiving signal from said sensors indicative of cardiac arrest.

11. The wearable defibrillator of claim 10, wherein said alarm comprises a sonic alarm.

12. The wearable defibrillator of claim 10, wherein said alarm comprises a tangible alarm.

13. The wearable defibrillator of claim 10, further comprises a user accessible abort switch.

14. The wearable defibrillator of claim 1, wherein said defibrillating subunit adapted to a defibrillation upon being activated from the remote location.

15. The wearable defibrillator of claim 1, further comprising a replaceable power source.

16. The wearable defibrillator of claim 1, further comprising a carrying subunit.

17. The wearable defibrillator of claim 16, wherein said carrying subunit is adapted to carry the wearable defibrillator.

18. The wearable defibrillator of claim 16, wherein said carrying subunit is adapted to position the wearable defibrillator on a user.

19. A method of defibrillating comprising:
sensing a cardiac related parameter;
sensing breathing; and
triggering a defibrillation energy upon determining a cardiac arrest (lack of breathing and lack of pulse).

20. The method of claim 19, wherein a cardiac related parameter comprises sensing mechanical or electrical pulse, obtaining an electrocardiogram (ECG) or a combination thereof.

21. The method of claim 19, wherein determining lack of effective breathing may be achieved using a sensed cardiac related parameter.

22. The method of claim 19, wherein defibrillation comprises administration of electrical defibrillation energy.

23. The method of claim 19, wherein said defibrillation comprises administration of mechanical defibrillation energy.

24. The method of claim 19, further comprising activating an alarm upon sensing a cardiac arrest.

25. The method of claim 19, further comprising manually inactivating the defibrillation upon indication of false alarm.

26. A wearable defibrillator comprising:
a first sensor adapted to sense a cardiac related paremeter;
a controller adapted to produce a signal upon determining a cardiac arrest;
a transmitter adapted to send a signal indicative of a cardiac arrest to a remote location; and a defibrillation subunit adapted to trigger a defibrillation upon being activated from the remote location.

27. The wearable defibrillator of claim 26, wherein said first sensor is adapted to sense mechanical or electrical pulse, obtain a electrocardiogram (ECG), or a combination thereof.

28. The wearable defibrillator of claim 27, wherein said first sensor comprises an electric sensor, an electromechanical sensor or both.

29. The wearable defibrillator of claim 28, wherein said electromechanical sensor comprises a piezo electric sensor.

30. The wearable defibrillator of claim 28, wherein said electromechanical sensor comprises strain gage sensor.

31. The wearable defibrillator of claim 28, wherein said electromechanical sensor comprises a pressure sensor.

32. The wearable defibrillator of claim 26, wherein said remote location include a health care center, a health care clinic, a hospital, an emergency station or any combination thereof.

33. The wearable defibrillator of claim 26, wherein said activation from remote location are transferred wirelessly.

34. The wearable defibrillator of claim 26, wherein said defibrillating energy is an electrical energy.

35. The wearable defibrillator of claim 26, wherein said defibrillating energy is a mechanical energy.

36. The wearable defibrillator of claim 26, further comprising an alarming subunit adapted to trigger an alarm upon receiving an alarm signal from a controller, wherein the controller is adapted to produce an alarm signal upon receiving signal from said sensor indicative of cardiac arrest.

37. The wearable defibrillator of claim 36, wherein said alarm comprises a sonic alarm.

38. The wearable defibrillator of claim 36, wherein said alarm comprises a tangible alarm.

39. The wearable defibrillator of claim 26, further comprises a user accessible abort switch.

40. The wearable defibrillator of claim 26, further comprising a power source.

41. The wearable defibrillator of claim 26, further comprising a carrying subunit.

42. The wearable defibrillator of claim 41, wherein said carrying subunit is adapted to carry the wearable defibrillator.

43. The wearable defibrillator of claim 41, wherein said carrying subunit is adapted to position the wearable defibrillator on a user.

44. A method of defibrillating comprising:
sensing a cardiac related parameter;
producing a signal upon determining a cardiac arrest;
transmitting a signal indicative of a cardiac arrest to a remote location; and
triggering a defibrillation upon being activated from the remote location.

45. The method of claim 44, wherein sensing cardiac related parameter comprises sensing mechanical or electrical pulse, obtaining a electrocardiogram (ECG), or a combination thereof.

46. The method of claim 44, further comprising sensing cardiac related parameter electrically, electromechanically or both.

47. The method of claim 44 wherein said remote location is a health care center, a health care clinic, a hospital, an emergency station or any combination thereof.

48. The method of claim 44, wherein said transmitting is by wireless communication.

49. The method of claim 44, wherein said defibrillation comprises administration of electrical defibrillation energy.

50. The method of claim 44, wherein said defibrillation comprises administration of mechanical defibrillation energy.

51. The method of claim 44, further comprising activating an alarm upon determining cardiac arrest.

52. The method of claim 44, further comprising manually inactivating the defibrillation upon indication of false alarm.
